(19) United States
(12) Patent Application Publication
(10) Pub. No.: US 2006/0251732 A1
(43) Pub. Date: Nov. 9, 2006

(54) Crystalloid electrolyte water formulation

(76) Inventors: Shawn D. Mueller, Fort Myers, FL (US); Michael M. Barlow, Bonita Springs, FL (US)

Correspondence Address: WALLENSTEIN & WAGNER, LTD., 311 SOUTH WACKER DRIVE, 53RD FLOOR, CHICAGO, IL, 60606, US

(21) Appl. No.: 11/121,443

(22) Filed: May 4, 2005

(51) Int. Cl. A61K 33/32; A61K 33/26; A61K 33/04; A61K 33/00; A61K 33/34; A61K 33/24; A61K 33/36

(52) U.S. Cl. 424/630; 424/641; 424/646; 424/656; 424/702; 424/722; 424/724; 424/639; 424/668

(57) ABSTRACT

A fortified liquid composition, comprising purified water, molecular oxygen at a level between 30 and 60 mg/l, and a combination of minerals in that purified water. The minerals include at least 0.002 mg/l boron, at least 0.0001 mg/l chromium, at least 0.045 mg/l cobalt, at least 0.019 mg/l copper, at least 0.086 mg/l iodine, at least 0.053 mg/l manganese, at least 0.36 mg/l potassium, at least 0.0003 mg/l selenium, at least 0.029 mg/l silica, at least 2.61 mg/l sodium, and at least 0.015 mg/l zinc.

### TECHNICAL FIELD

The invention relates to a performance water drink that is enriched with a blend of crystalloid electrolytes and, optionally, oxygen.

### BACKGROUND OF THE INVENTION

Electrolytes are micronutrients that are necessary components of the human diet. Electrolytes are mineral salts, in ionic form. Electrolytes help to maintain voltages across nerve, heart, muscle, and other body cell membranes. Electrolytes also facilitate the transport of electrical impulses, such as nerve impulses and muscle contractions, across nerve and muscle cells, respectively.

Electrolytes also influence the pH of the human body. These micronutrients play an important role in energy production, hemoglobin synthesis, and the maintenance of bone health. Furthermore, electrolytes are important in maintaining the health of the immune system, in protecting body tissues from oxidative damage, and in building and repairing muscle tissue after exercise.

Electrolytes are formed when minerals come together in solution. That solution provides energy for the body. Anions or cations, which are formed upon the dissolution of the electrolytes, facilitate the transport of the electrical impulses.

Electrolytes also facilitate delivery of oxygen within the body, to help maintain peak brain function and nervous system response.

In summary, electrolytes are important for human health. Thus, it is important that humans have both adequate supplies of electrolytes, and effective means of delivering such electrolytes to the body.

A prepackaged electrolyte-containing solution is sold under the brand name TraceLyte. TraceLyte is a product of Nature's Path, and is available through conventional and internet retailers. TraceLyte includes eleven minerals. Its label discloses that TraceLyte can be added to water to create a fortified drink. Moreover, a website sponsored by Nature's Path discloses the use of TraceLyte with water: That website states, near the bottom of the first page, under “How to Use”, the following: “as a dietary supplement for adults, take 1 teaspoon diluted in eight (8) oz. of water three times daily . . . ”

This dosage calculates as a relatively concentrated dosage, i.e., 0.0208 parts TraceLyte per 1 part of water.

The present invention is a novel and effective means of rapidly delivering both oxygen and essential electrolytes to the body, using highly purified water. The present invention accomplishes this at substantially smaller dosages than those disclosed in the prior art.

### SUMMARY OF THE INVENTION

The invention is a fortified liquid composition, comprising purified water, and a combination of minerals in that purified water, the minerals comprising boron, chromium, cobalt, copper, iodine, manganese, potassium, selenium, silica, sodium, and zinc. More particularly, the fortified liquid composition comprises: (a) at least 0.002 mg/l boron; (b) at least 0.0001 mg/l chromium; (c) at least 0.045 mg/l cobalt; (d) at least 0.019 mg/l copper; (e) at least 0.086 mg/l iodine; (f) at least 0.053 mg/l manganese; (g) at least 0.36 mg/l potassium; (h) at least 0.0003 mg/l selenium; (i) at least 0.029 mg/l silica; (j) at least 2.61 mg/l sodium; (k) at least 0.015 mg/l zinc.

The fortified liquid composition is further enriched with molecular oxygen, such that the level of infused oxygen in the composition is 30 to 60 mg/l.

Other features and advantages of the invention will be apparent from the following specification taken in conjunction with the following drawings.

### BRIEF DESCRIPTION OF THE DRAWINGS

To understand the present invention, it will now be described by way of example, with reference to the accompanying drawings in which:

FIG. 1 is a set of photographs showing the red blood cells of Test Subject #1, both before and after the ingestion of the fortified liquid composition of the invention.

FIG. 2 is a set of photographs showing the red blood cells of Test Subject #2, both before and after the ingestion of the fortified liquid composition of the invention.

### DETAILED DESCRIPTION OF A PREFERRED EMBODIMENT

This invention is susceptible of embodiments in many different forms. The preferred embodiment of the invention is to be considered as but one example of the principles of the invention. The preferred embodiment is not intended to limit the broadest aspect of the invention to the embodiment described in this specification

The invention is a fortified liquid composition. In essence, the fortified liquid composition is a combination of water, eleven minerals, and infused oxygen.

The water component of the liquid composition is a highly purified water. The water component is preferably made in a multi-step purification process.

In order to make the water component, standard tap water is subjected to a multi-step purification process. First, that tap water is softened by passage through a cationic ion exchange water softener.

Two filtration steps are then utilized. First, the water is filtered through bacteriostatic silver-impregnated carbon media. Second, the water is subjected to five (5) micron sediment filtration.

The water is next subjected to reverse osmosis, ultraviolet sterilization, and ozonation.

The water next undergoes an ion exchange polishing step, using ultra pure mixed bed resins. The water then is filtered in an absolute sub micron (0.2 micron) filtration step.

While the above description provides on way of attaining the highly purified water used in this preferred embodiment of the invention, there are other well-known methods of attaining water of this quality level. Any of those methods may be used to prepare the water used with this preferred embodiment.

Regardless of the method used, the purified, processed water should: (a) meet or exceed the USP-23 quality standard; and (b) have a specific conductivity of at least 1-2 μS/cm.

After the water has been treated to meet these quality standards, it is ready for blending with the minerals. The eleven (11) minerals used to make the fortified liquid composition of the invention include copper, iodine, manganese, zinc, potassium, cobalt, sodium, selenium, chromium, silica, and boron. In order to provide the beneficial effects of the fortified water, these eleven elements should be provided in certain minimum amounts.

For the purposes of this invention, the minimum amounts of these minerals are as follows: (a) at least 0.002 mg/l boron; (b) at least 0.0001 mg/l chromium; (c) at least 0.045 mg/l cobalt; (d) at least 0.019 mg/l copper; (e) at least 0.086 mg/l iodine; (f) at least 0.053 mg/l manganese; (g) at least 0.36 mg/l potassium; (h) at least 0.0003 mg/l selenium; (i) at least 0.029 mg/l silica; (j) at least 2.61 mg/l sodium; (k) at least 0.015 mg/l zinc.

It will be understood, however, that these minima are not exact. There is a tolerance in the amounts of these minerals. Particularly, for the purposes of this invention, the minimum amount of boron is 0.002 mg/l±4%; the minimum amount of chromium is 0.0001 mg/l±3%; the minimum amount of cobalt is 0.045 mg/l±3%; the minimum amount of copper is 0.019 mg/l±5%; the minimum amount of iodine is 0.086 mg/l±3%; the minimum amount of manganese is 0.053 mg/l±6%; the minimum amount of potassium is 0.36 mg/l±7%; the minimum amount of selenium is 0.0003 mg/l±3%; the minimum amount of silica is 0.029 mg/l±5%; the minimum amount of sodium is 0.2.61 mg/l±7%; and the minimum amount of zinc is 0.015 mg/l±3%.

As noted above, the fortified liquid composition of the invention is primarily made of water and a specific combination of eleven minerals. In order to make the composition of the invention, approximately 1,500 gallons of the purified water is added to a composition that includes the eleven (11) minerals. In particular, the 1,500 gallons of the purified water is added to thirty-five (35) gallons of TraceLyte. TraceLyte is a concentrate that is manufactured by a company known as Nature's Path. TraceLyte includes all eleven of the minerals necessary for the manufacture of the fortified liquid composition of the invention.

After thirty-five (35) gallons of TraceLyte is added to 1,500 gallons of the purified water, additional water is added, until the total amount of liquid reaches 3,000 gallons. The TraceLyte and the purified water are mixed, for example, by recirculation with the pump or pumps that fill the container in which the TraceLyte and water are being blended.

In particular, the addition of thirty-five (35) gallons of TraceLyte to approximately 2,965 gallons of purified water results in a fortified liquid composition of the invention, and in particular a fortified liquid composition having approximately 0.002 mg/l boron; 0.0001 mg/l chromium; 0.045 mg/l cobalt; 0.019 mg/l copper; 0.086 mg/l iodine; 0.053 mg/l manganese; 0.36 mg/l potassium; 0.0003 mg/l selenium; 0.029 mg/l silica; 2.61 mg/l sodium; and 0.015 mg/l zinc.

In contrast, another embodiment of the invention can be made by the addition of fifty-two and one-half (52.5) gallons of TraceLyte to approximately 2,947.5 gallons of purified water. In particular, this fortified liquid composition has approximately 0.003 mg/l boron; 0.00015 mg/l chromium; 0.067 mg/l cobalt; 0.027 mg/l copper; 0.129 mg/l iodine; 0.079 mg/l manganese; 0.54 mg/l potassium; 0.00045 mg/l selenium; 0.043 mg/l silica; 3.91 mg/l sodium; and 0.022 mg/l zinc.

The final step of the manufacture of the fortified liquid composition of the invention comprises enriching the water with molecular oxygen through a super oxygenation process. This oxygenation step is done immediately prior to bottling. Preferably, the water is infused with oxygen to an extent that the final fortified liquid composition includes 30 to 60 mg/l of oxygen.

By infusing the water with 30-60 mg of molecular oxygen provides that water with optimal balance and stability. Electrolytes facilitate delivery of oxygen to achieve and maintain peak brain function, and proper nervous system response.

The constant firing of micro-electric impulses across the synapses of the brain requires a great deal of energy. Only electrolytes can supply this energy. If, as the result of an electrolyte imbalance, an inadequate amount of oxygen is available for the firing of the nerve cells, then the brain functions less effectively.

In addition, the body uses oxygen to turn nutrients into energy, through the process of primary oxygenation. In other words, the electrolytes help the oxygen create a chemical reaction that ultimately allows the body to “burn” the nutrients as fuel.

In summary, bio-oxidation liberates energy, which facilitates life. In this invention, these biological occur very rapidly. It is believed that this rapid response arises due to the proprietary, molecular size of the electrolytes in crystalloid form. The molecular size of both the crystalloid electrolytes and the oxygen, after the infusion process, is believed to remain small enough to penetrate the red blood cells, inducing nearly immediate results.

The overall size of each infused molecular structure of this soluble invention remains minute enough to penetrate the soft tissues of the pallet, esophagus and stomach upon contact. The molecules thus are believed to move immediately into the bloodstream, actively influencing every red blood cell in the body.

After the fortified liquid composition has been oxygenated in the manner described above, the final product may be bottled. Preferably, the water is bottled in twenty-ounce polyethylene terpthalate (PET) bottles, and then capped. This fortified liquid composition is ready for human consumption.

The fortified liquid composition of the present invention has been demonstrated to have extremely fast acting and beneficial effects on the user. These beneficial effects can be established by a clinical testing protocol known as phase contrast microscopy. Phase contrast microscopy is a live blood analysis used to asses the overall health, hydration, and electromagnetic energy levels of the erythrocytes of human test subject.

In accordance with the protocol, a physician and an individual are seated side-by-side. The individual upon whom the test will be performed is free of known diseases, or symptoms of such diseases. The attending physician ensures a sterile environment, wears surgical gloves, and uses a disposable lancet. The individual upon whom the test is conducted has refrained from consumption of all food and beverages (except for commercially produced drinking water) for at least ten (10) hours prior to the test.

The testing physician chooses a finger of the test subject, and thoroughly cleanses that finger tip with a disposable medical grade alcohol swab.

When the finger has dried, the test finger is perforated with a single use lancet. Upon perforation, the testing physician extracts a single drop of blood and carefully places it upon a laboratory approved slide cover.

The testing physician places the laboratory slide cover under a binocular, compound microscope with oil emulsion magnification capabilities. The microscope is interfaced with a color monitor to permit immediate observation.

The test subject is provided with a cotton ball, and instructed to place that cotton ball on the perforation site to stop the flow of blood.

The testing physician begins to assess the condition of the blood, both verbally and through photographs captured on computer software that interfaces between the microscope and the monitor. The blood is assessed for the rate of active flow within the plasma, and the level of osmotic pressure within the red blood cells. This level corresponds to the level of true hydration within the red blood cells, and to the visual level of electromagnetic energy in the cells. Electromagnetic resonance as it pertains to the erythrocytes is indicated by the presence of a visible white ring encircling the outer perimeter of the red blood cells.

At this point, this same test subject ingests, over a very short time period (e.g., three to seven seconds), 10-12 ounces of the fortified liquid composition described above.

Ninety seconds after the ingestion of that fortified liquid composition, the physician repeats the exact procedure described above for drawing and observing the subject's blood.

The testing physician repeats the observations of the characteristics of the blood sample.

This testing established that the ingestion of the fortified liquid composition in accordance with the invention hydrates and induces a strong and noticeable electromagnetic presence within and upon the red blood cells. This beneficial effect on the blood takes place in as little as sixty to ninety seconds after the ingestion of the fortified liquid composition.

It is believed that this rapid effect results from the fact that the electrolytes and oxygen in the fortified liquid composition are small enough to readily and rapidly penetrate the erythrocytes (or red blood cell) wall. As a result, in a matter of seconds, a powerful boost in energy is imparted to the cells, and active nutrition is provided to the subject's body.

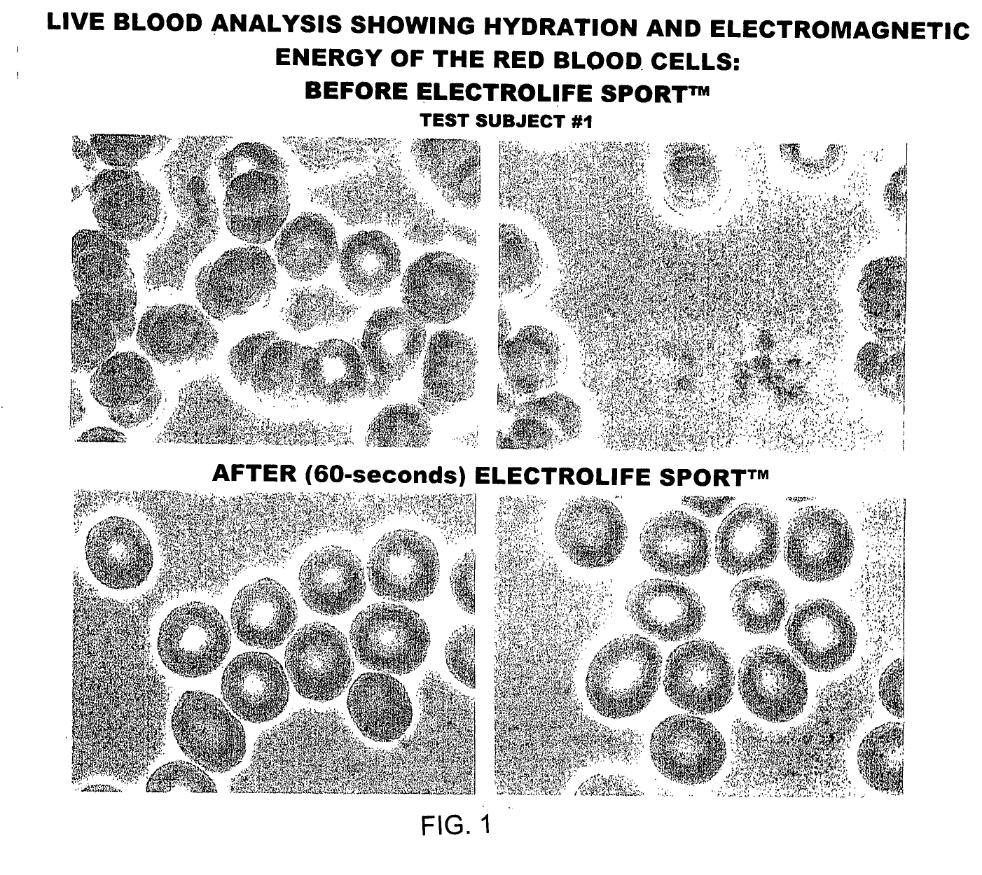

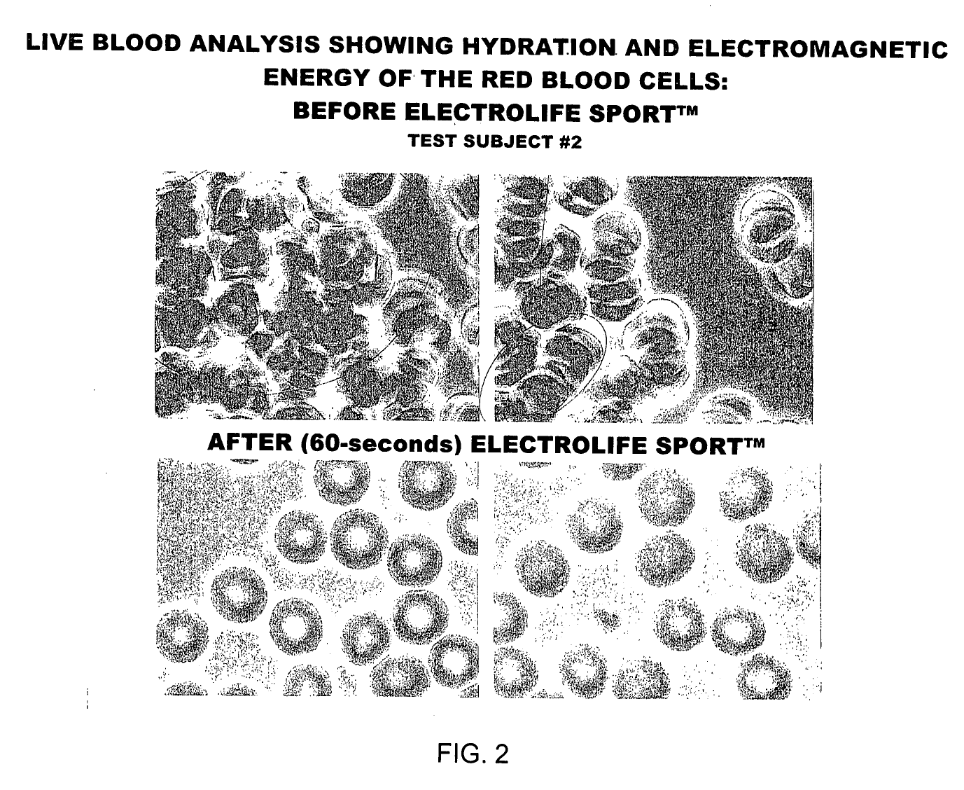

The effects can be seen in FIGS. 1 and 2. FIG. 1 is a set of photographs showing the red blood cells of Test Subject #1. The two photographs at the top of FIG. 1 depict the red blood cells before ingestion of the fortified liquid composition of the invention, and the two photographs at the bottom of FIG. 1 depict the red blood cells of the same individual sixty seconds after the ingestion of the fortified liquid composition of the invention. It can be clearly seen that after ingestion of the fortified liquid compositions of the invention, the blood cells of Subject #1 show evidence of dramatically increased hydration and electromagnetic energy.

FIG. 2 is a set of photographs showing the red blood cells of Test Subject #2. The two photographs at the top of FIG. 2 depict the red blood cells before ingestion of the fortified liquid composition of the invention, and the two photographs at the bottom of FIG. 2 depict the red blood cells of the same individual sixty seconds after the ingestion of the fortified liquid composition of the invention. Again, it can be clearly seen that after ingestion of the fortified liquid compositions of the invention, the blood cells of Subject #2 show evidence of dramatically increased hydration and electromagnetic energy.

The present invention creates a fortified liquid composition having a concentration of about thirty-five gallons TraceLyte per 2,965 gallons water, i.e., about 0.0118 parts TraceLyte per 1 part of water. Another aspect of the present invention creates a fortified liquid composition having a concentration of about fifty-two and one-half (52.5) gallons TraceLyte per 2,947.5 gallons water, i.e., about 0.0177 parts TraceLyte per 1 part of water. In contrast, the Nature's Path website provides that a substantially greater amount of TraceLyte should be used, i.e., 0.0208 parts TraceLyte per 1 part water.

Accordingly, the present invention is very effective in hydrating and oxygenating the blood of the user, at substantially lower levels than the levels suggested in the prior art.

While the specific embodiments have been illustrated and described, numerous modifications come to mind without significantly departing from the spirit of the invention. Thus, the scope of protection is only limited by the scope of the accompanying Claims.

What is claimed is:

1. A fortified liquid composition, consisting essentially of water, molecular oxygen at a level between 30 and 60 mg/l, and the minerals copper, iodine, manganese, zinc, potassium, cobalt, sodium, selenium, chromium, silica, and boron.

2. A fortified liquid composition, comprising purified water, and a combination of minerals in that purified water, the minerals comprising: (a) 0.002-0.003 mg/l boron; (b) 0.0001-0.00015 mg/l chromium; (c) 0.045-0.067 mg/l cobalt; (d) 0.019-0.027 mg/l copper; (e) 0.086-0.129 mg/l iodine; (f) 0.053-0.079 mg/l manganese; (g) 0.36-0.54 mg/l potassium; (h) 0.0003-0.00045 mg/l selenium; (i) 0.029-0.043 mg/l silica; (j) 2.61-3.91 mg/l sodium; (k) 0.015-0.022 mg/l zinc.

3. The fortified liquid composition of claim 2, said composition being further enriched with molecular oxygen, such that the level of oxygen in the composition is between 30 and 60 mg/l.

4. A fortified liquid composition, comprising purified water, molecular oxygen at a level between 30 and 60 mg/l, and a combination of minerals in that purified water, the minerals comprising: (a) at least 0.002 mg/l boron; (b) at least 0.0001 mg/l chromium; (c) at least 0.045 mg/l cobalt; (d) at least 0.019 mg/l copper; (e) at least 0.086 mg/l iodine; (f) at least 0.053 mg/l manganese; (g) at least 0.36 mg/l potassium; (h) at least 0.0003 mg/l selenium; (i) at least 0.029 mg/l silica; (j) at least 2.61 mg/l sodium; (k) at least 0.015 mg/l zinc.
